(19) United States
(12) Patent Application Publication
(10) Pub. No.: US 2023/0021062 A1
(43) Pub. Date: Jan. 19, 2023

(54) BELT AND ELECTROCARDIOGRAPHIC MEASUREMENT DEVICE

(71) Applicant: OMRON HEALTHCARE Co., Ltd., Kyoto (JP)

(72) Inventors: Yuki SAKAGUCHI, Kyoto (JP); Yasuhiro KAWABATA, Kyoto (JP); Kenji FUJII, Kyoto (JP); Naomi MATSUMURA, Kyoto (JP); Reiji FUJITA, Kyoto (JP); Akito ITO, Kyoto (JP); Nozomi KAWAI, Kyoto (JP)

(21) Appl. No.: 17/934,929

(22) Filed: Sep. 23, 2022

Related U.S. Application Data
(63) Continuation of application No. PCT/JP2021/007619, filed on Mar. 1, 2021.

(30) Foreign Application Priority Data
Mar. 31, 2020 (JP) 2020-062900

(51) Int. Cl. A61B 5/282; A61B 5/00; A61B 5/273

(52) U.S. Cl. CPC A61B 5/282; A61B 5/6831; A61B 5/273

(57) ABSTRACT

A belt used in an electrocardiographic measurement apparatus includes a belt body to be wrapped around a living body, three or more base electrodes disposed in a longitudinal direction of the belt body, and two or more cap electrodes detachable and attachable to the base electrodes and smaller in number than the number of base electrodes.

### CROSS-REFERENCE TO RELATED APPLICATIONS

This application is the U.S. national stage application filed pursuant to 35 U.S.C. 365(c) and 120 as a continuation of International Patent Application No. PCT/JP2021/007619, filed Mar. 1, 2021, which application claims priority to Japanese Patent Application No. 2020-062900, filed Mar. 31, 2020, which applications are incorporated herein by reference in their entireties.

### TECHNICAL FIELD

The present invention relates to a belt and an electrocardiographic measurement apparatus that are used in measurement of a biological signal in accordance with an electric potential generated due to cardiac motion on the surface of a living body.

### BACKGROUND ART

An electrocardiographic measurement apparatus is known that detects, as one of biological signals, an electrocardiographic signal, which is a voltage generated due to cardiac motion on the surface of a living body, and generates an electrocardiographic waveform of a user.

Patent Document 1 discloses, as such an electrocardiographic measurement apparatus, an electrocardiographic measurement apparatus that uses a belt including a belt body to be wrapped around the upper arm of a user and a plurality of electrodes fixed to an inner surface of the belt body at equal intervals in one direction.

### CITATION LIST-PATENT LITERATURE

Patent Document 1: JP 5428889 B

### SUMMARY OF INVENTION

### Technical Problem

In the electrocardiographic measurement apparatus described above, the number of the plurality of electrodes disposed on the belt is increased to accommodate the upper arms of a variety of users. In other words, the circumferential length of the upper arm differs depending on the users. Accordingly, the belt of the electrocardiographic measurement apparatus requires a large number of electrodes to accommodate the upper arms having different circumferential lengths. In addition, when the number of electrodes is increased, an electrocardiographic waveform is formed based on an output of each of the electrodes, and thus a circuit and processing are complicated.

It is also conceivable to reduce the number of electrodes. However, the electric potential distribution generated due to cardiac motion on the living body surface varies between individuals, and when an electrocardiographic waveform is measured with a small number of electrodes, the electric potential (electrocardiographic signal) detection intensity for each user varies depending on the circumferential length of the upper arm. Therefore, a technique is required that can suitably detect an electrocardiographic signal with a small number of electrodes, for example, by suppressing the difference in electrocardiographic signal detection intensity due to the individual difference in electric potential distribution on the living body surface.

An object of the present invention is thus to provide a belt and an electrocardiographic measurement apparatus that can suitably detect an electrocardiographic signal with a small number of electrodes.

### Solution to Problem

According to an aspect of the present invention, a belt is provided that includes: a belt body to be wrapped around a living body; three or more base electrodes disposed in a longitudinal direction of the belt body; and two or more cap electrodes detachable and attachable to the base electrodes and smaller in number than the number of base electrodes.

According to this aspect, the cap electrodes can be selectively attached to the plurality of base electrodes. Therefore, the cap electrodes can be disposed at suitable positions with respect to the distribution of electric potentials on the living body surface, which are generated due to cardiac motion of a subject on which electrocardiographic measurement is performed. As a result, an electrocardiographic signal can be suitably detected with a small number of electrodes. For example, by arranging the cap electrode at a position in which the electrocardiographic signal detection intensity is high, the intensity of an electrocardiographic signal to be detected can be enhanced.

According to the belt of the aspect described above, the belt is provided in which the base electrodes are fixed to the belt body by affixing, sewing, inserting, crimping, or magnetic force, and the cap electrodes are fixed to the base electrodes selectively and detachably and attachably by inserting, magnetic force, or screwing.

According to this aspect, since the base electrodes are fixed to the belt body, only the cap electrodes can be selectively mounted on the base electrodes. Therefore, in order to change the electrode to be brought in contact with the living body, the cap electrode only need to be shifted in the belt.

According to the belt of the aspect described above, the belt is provided in which the cap electrodes include surfaces that are brought into contact with the living body, the surfaces being formed in a circular shape or a polygonal shape.

According to this aspect, the cap electrode that is brought into contact with the living body can be formed in a shape suitable for coming into contact with the living body.

According to the belt of the aspect described above, the belt is provided in which one of the base electrode and the cap electrode includes a recess, and another of the base electrode and the cap electrode includes an insertion portion to be inserted into the recess.

According to this aspect, by inserting one of the base electrode and the cap electrode into the other, the base electrode and the cap electrode can be fixed.

According to the belt of the aspect described above, the belt is provided in which the recess and the insertion portion are shaped to restrict rotation of the insertion portion around an axis of the insertion portion.

According to this aspect, the rotation of the cap electrode can be suppressed. Therefore, for example, even when the surface of the cap electrode, which is brought into contact with the living body is formed in a shape that comes into contact with the adjacent cap electrode when the cap electrode rotates, the contact between the cap electrodes can be suppressed.

According to the belt of the aspect described above, the belt is provided in which the cap electrode is a dry electrode or a wet electrode.

According to this aspect, when the cap electrode detects an electrocardiographic signal, the suitable electrode can be provided.

An electrocardiographic measurement apparatus is provided that includes: the belt according to the aspect described above; and a device body configured to detect an electrocardiographic waveform by the cap electrodes mounted on the base electrodes.

According to this aspect, the electrocardiographic waveform can be detected by using the belt provided with the cap electrodes that are selectively disposed in each belt depending on a subject.

### Advantageous Effects of Invention

According to an aspect of the present invention, a belt and an electrocardiographic measurement apparatus that can suitably detect an electrocardiographic signal with a small number of electrodes can be provided.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1 is an explanatory diagram illustrating the configuration of an electrocardiographic measurement apparatus according to a first embodiment of the present invention;

FIG. 2 is a plan view illustrating the configuration of the electrocardiographic measurement apparatus;

FIG. 3 is a block diagram illustrating the configuration of the electrocardiographic measurement apparatus;

FIG. 4 is a cross-sectional view illustrating, partially omitted, the configuration a belt used in the electrocardiographic measurement apparatus;

FIG. 5 is a plan view illustrating, partially omitted, the configuration of the belt;

FIG. 6 is a plan view illustrating, partially omitted, the configuration of the belt;

FIG. 7 is an explanatory diagram illustrating, assuming that the cross-sectional shape of the upper arm is circle, the arrangement of the first electrode to the ninth electrode;

FIG. 8 is a graph illustrating an example of time series variations of electrocardiographic waveforms detected by respective pairs of electrodes;

FIG. 9 is a graph illustrating an example of time series variations of electrocardiographic waveforms detected by respective pairs of each electrode and a ground electrode in different subjects;

FIG. 10 is an explanatory diagram illustrating an example of the distance between two electrodes on the upper arm;

FIG. 11 is an explanatory diagram illustrating a histogram of the distance between the electrodes where the potential difference is largest;

FIG. 12 is a cross-sectional view illustrating, partially omitted, the configuration of a belt according to a second embodiment of the present invention;

FIG. 13 is a cross-sectional view illustrating, partially omitted, the configuration of a belt according to a third embodiment of the present invention;

FIG. 14 is a cross-sectional view illustrating, partially omitted, the configuration of a belt according to a fourth embodiment of the present invention;

FIG. 15 is a plan view illustrating, partially omitted, the configuration of a belt according to a fifth embodiment of the present invention;

FIG. 16 is a plan view illustrating, partially omitted, the configuration of a belt according to a sixth embodiment of the present invention;

FIG. 17 is a cross-sectional view illustrating, partially omitted, the configuration of a belt according to a seventh embodiment of the present invention;

FIG. 18 is a plan view illustrating, partially omitted, the configuration of a belt according to another embodiment of the present invention;

FIG. 19 is a plan view illustrating, partially omitted, the configuration of a belt according to another embodiment of the present invention;

FIG. 20 is a cross-sectional view illustrating, partially omitted, the configuration of a belt according to another embodiment of the present invention; and,

FIG. 21 is a cross-sectional view illustrating, partially omitted, the configuration of a belt according to another embodiment of the present invention.

### DESCRIPTION OF EMBODIMENTS

Embodiments according to one aspect of the present invention will be described below based on the drawings. Note that the embodiments described below are merely illustrative of the present invention in all respects.

### First Embodiment

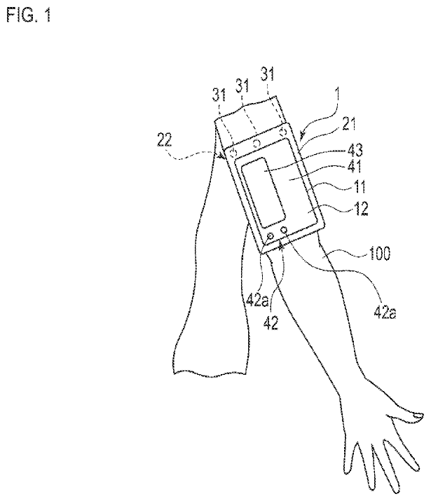

Herein, an example of an electrocardiographic measurement apparatus 1 and a belt 11 according to a first embodiment of the present invention will be described below with the use of FIGS. 1 to 7.

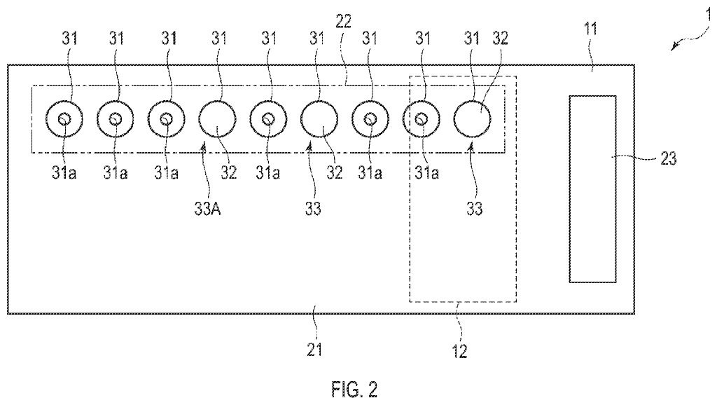

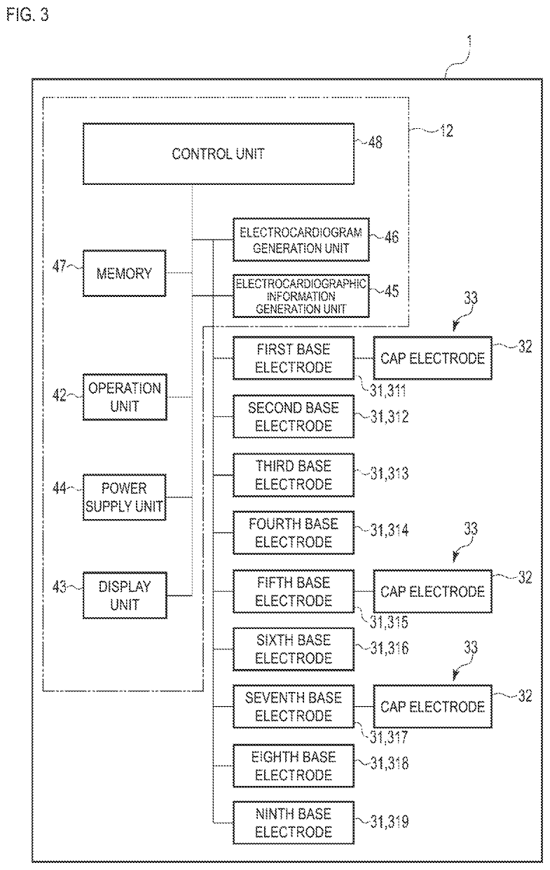

FIG. 1 is an explanatory diagram illustrating the configuration of the electrocardiographic measurement apparatus 1 according to the first embodiment of the present invention and illustrating a state where the electrocardiographic measurement apparatus 1 is attached to an upper arm 100 of a living body. FIG. 2 is a developed plan view illustrating, from the living body side, the configuration of the electrocardiographic measurement apparatus 1. FIG. 3 is a block diagram illustrating the configuration of the electrocardiographic measurement apparatus 1.

The electrocardiographic measurement apparatus 1 is an electric potential measurement apparatus that is attached to the living body to detect electric potentials at a plurality of points on the skin surface of a living body and generate electrocardiographic information necessary for generating an electrocardiogram based on the detected electric voltage. Note that the electrocardiographic measurement apparatus 1 may generate an electrocardiographic waveform and display the electrocardiographic waveform and may be configured to display information necessary for generating an electrocardiogram and output the information to an external terminal.

As illustrated in FIGS. 1 and 2, the electrocardiographic measurement apparatus 1 includes the belt 11 and a device body 12. In the electrocardiographic measurement apparatus 1, for example, the belt 11 and the device body 12 are integrally formed. The electrocardiographic measurement apparatus 1 functions as, for example, a so-called wearable device to be attached by the belt 11 to the upper arm as the living body. FIG. 1 illustrates an example of a state where the electrocardiographic measurement apparatus 1 is attached to the upper arm 100 of the subject. Note that the electrocardiographic measurement apparatus 1 may include the belt 11 and the device body 12 that are configured as separate components and are connected via a signal line or the like.

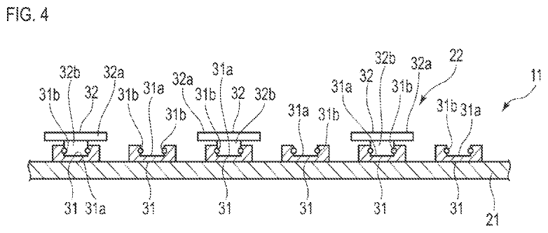

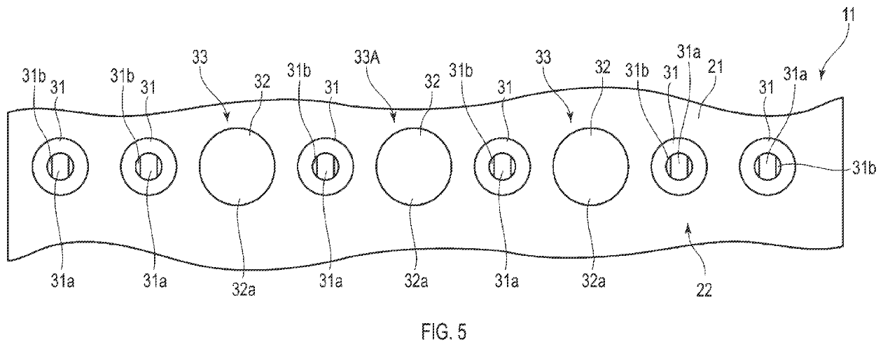

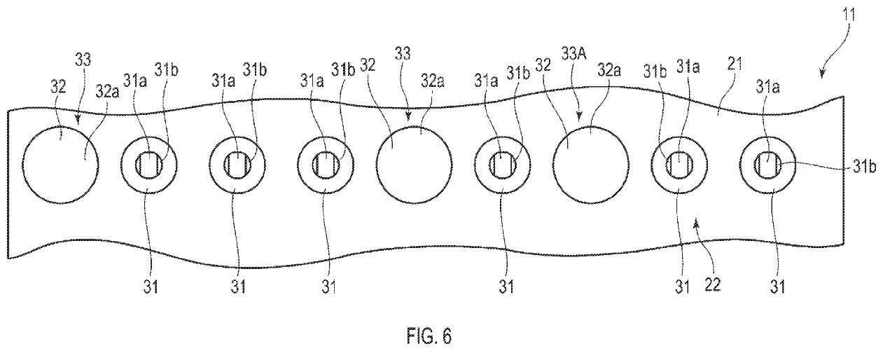

The belt 11 holds the device body 12. The belt 11 is wrapped around the living body. The belt 11 is attached, for example, to the upper arm of the subject as illustrated in FIG. 1. As illustrated in FIGS. 2 and 4 to 6, the belt 11 includes a belt body 21, an electrode array 22, and a fixing means 23. Note that the belt 11 is illustrated with a portion of the belt body 21, a portion of the configuration of the electrode array 22, and the fixing means 23 omitted in FIG. 4 and with a portion of the belt body 21 and the fixing means 23 omitted in FIGS. 5 and 6.

The belt body 21 is formed of, for example, a resin or a fiber having flexibility. The belt body 21 is set to have a length that can be attached to the upper arm of the subject wearing the electrocardiographic measurement apparatus 1. The belt body 21 is formed in a band shape that is long in one direction. The belt body 21 is configured such that when the electrocardiographic measurement apparatus 1 is attached to the upper arm, the device body 12 is fixed to the surface, which is the outer surface and the electrode array 22 is disposed on the back surface, which is the surface on the living body side.

The electrode array 22 is electrically connected via a signal line or the like to the device body 12. The electrode array 22 includes a plurality of base electrodes 31 and a plurality of cap electrodes 32.

The plurality of base electrodes 31 are disposed side by side at equal intervals in a longitudinal direction of the belt body 21. Each of the plurality of base electrodes 31 is electrically connected to the device body 12. The base electrode 31 is formed to enable the cap electrode 32 to be attached and detached.

The number of base electrodes 31 can be appropriately set to the number that can generate an electrocardiographic waveform even when the upper arms of subjects have different arm circumference lengths. In other words, in order to accommodate the upper arms of a variety of subjects, the number of base electrodes 31 is set to the number of electrodes required to generate an electrocardiographic waveform, specifically two or more.

In the present embodiment, an example in which nine base electrodes 31 are disposed is described.

The cap electrode 32 is detachably and attachably formed on the base electrode 31. The cap electrodes 32 are used to detect an electrocardiographic signal for generating an electrocardiographic waveform with the number of cap electrodes 32 smaller than the number of base electrodes 31. In other words, the cap electrode 32 having the number, specifically, at least two, that can detect an electrocardiographic signal necessary to generate an electrocardiographic waveform are disposed. For example, three cap electrodes 32 may be disposed. In this case, two cap electrodes 32 are used for the electrodes 33 that detect electric potentials on the skin surface of the upper arm. Also, one cap electrode 32 forms a ground electrode 33A. Such three cap electrodes 32 are selectively mounted on nine base electrodes 31 such that when the electrocardiographic measurement apparatus 1 is attached to the upper arm of the subject, the electrocardiographic signal detected from the subject is a strong signal.

The electrodes 33, 33A that detect the electrocardiographic signal are formed by the base electrodes 31 and the cap electrodes 32 mounted on the base electrodes 31.

Specific examples of such base electrodes 31 and cap electrodes 32 are described below. In order to allow the cap electrode 32 to be detachably and attachably mounted on the base electrode 31, the base electrode 31 and the cap electrode 32 are insertably formed. FIGS. 2, 5 and 6 illustrate examples of different arrangements of the cap electrodes 32 mounted on the base electrodes 31.

For example, one of the base electrode 31 and the cap electrode 32 includes a spring, and the other is formed in a snap button shape including a stud (projected shape).

As a specific example, as illustrated in FIGS. 4 to 6, the base electrode 31 includes a recess 31a and a spring 31b disposed in the recess 31a. The base electrodes 31 are fixed to the belt body 21 by affixing, sewing, inserting, crimping, or magnetic force. In FIG. 4, an example in which the base electrodes 31 are fixed to the belt body 21 by affixing or sewing is illustrated.

The cap electrode 32 is, for example, a dry electrode. As illustrated in FIGS. 4 to 6, the cap electrode 32 includes a tabular electrode portion 32a that is brought into contact with a living body and a stud 32b that is an insertion portion inserted into the recess 31a. The electrode portion 32a includes a surface that is brought into contact with the living body, and the surface is formed, for example, in a circular planer shape. The stud 32b is a projection and is inserted into the recess 31a of the base electrode 31 and held by the spring 31b disposed in the recess 31a, therefore being selectively fixed to the base electrode 31.

The fixing means 23 fixes the belt body 21 with the belt body 21 wrapped around the upper arm. The fixing means 23 is, for example, a hook-and-loop fastener. The hook-and-loop fastener includes a loop surface member and a hook surface member that are fixed respectively to the front surface side and the back surface side of the belt body 21. Note that a region disposed in the belt body 21, or the like of the loop surface member and the hook surface member can be appropriately set. By using such a hook-and-loop fastener, the fixing means 23 can fix the belt body 21 to the upper arm of the subject with the belt body 21 wound in the circumferential direction of the upper arm.

The device body 12 includes a case 41, an operation unit 42, a display unit 43, a power supply unit 44, an electrocardiographic information generation unit 45, an electrocardiogram generation unit 46, a memory 47, and a control unit 48.

Further, the device body 12 includes a communication unit that transmits and receives information with an external terminal. Note that the communication unit transmits and receives information with an external terminal wirelessly and/or in a wired manner.

The case 41 houses a portion of the operation unit 42, a portion of the display unit 43, the electrocardiographic information generation unit 45, the electrocardiogram generation unit 46, the memory 47, and the control unit 48. Additionally, the case 41 exposes a portion of the operation unit 42 and a portion of the display unit 43 from the outer surface. The case 41 is fixed to the belt 11.

The operation unit 42 is configured to receive an instruction input from a user. For example, the operation unit 42 includes a plurality of buttons 42a and a sensor that detects operations of the buttons 42a. Note that the operation unit 42 may include a touch panel of a pressure sensitive type, a capacitance type, or the like, a microphone that receives an instruction by sound, or the like, which are disposed on the case 41, the display unit 43, or the like. When operated by the user, the operation unit 42 converts the instruction into an electrical signal and outputs the electrical signal to the control unit 48.

The display unit 43 is electrically connected to the control unit 48. The display unit 43 is, for example, a liquid crystal display (LCD) or an organic electro luminescence display (OELD). The display unit 43 displays date and time, electrocardiographic information, an electrocardiographic waveform, or the like in accordance with a control signal from the control unit 48. Note that, in a case where the electrocardiographic measurement apparatus 1 is used in a biological information measurement device that displays a blood pressure value, the display unit 43 may display a variety of information including measurement results of blood pressure values such as systolic blood pressure and diastolic blood pressure, a heart rate, or the like.

The power supply unit 44 is a power source. The power supply unit 44 is, for example, a rechargeable battery such as a lithium ion battery. The power supply unit 44 is electrically connected to the control unit 48. As a specific example, the power supply unit 44 supplies power to the control unit 48. The power supply unit 44 supplies power for drive to the control unit 48, and via the control unit 48 to the operation unit 42, the display unit 43, the electrocardiographic information generation unit 45, the electrocardiogram generation unit 46, and the memory 47.

The electrocardiographic information generation unit 45 is electrically connected, for example, via a signal line to the plurality of base electrodes 31 of the electrode array 22. The electrocardiographic information generation unit 45 calculates a potential difference from voltages detected by the two cap electrodes 32. Specifically, the electrocardiographic information generation unit 45 calculates a potential difference between the two cap electrodes 32 mounted on two base electrodes 31 of the nine base electrodes 31 and generates electrocardiographic information.

The electrocardiogram generation unit 46 is electrically connected to the electrocardiographic information generation unit 45. The electrocardiogram generation unit 46 generates information of an electrocardiogram based on the electrocardiographic information generated by the electrocardiographic information generation unit 45. The information of the electrocardiogram may include electrocardiographic waveforms.

The electrocardiographic information generation unit 45 and the electrocardiogram generation unit 46 as just described are processing circuits that can respectively execute, for example, functions of the electrocardiographic information generation unit 45 and the electrocardiogram generation unit 46. The electrocardiographic information generation unit 45 and the electrocardiogram generation unit 46 are electrically connected to the control unit 48. Note that the control unit 48 includes the processing circuits of the electrocardiographic information generation unit 45 and the electrocardiogram generation unit 46 and executes a program stored in the memory 47 and thus may perform the functions of the electrocardiographic information generation unit 45 and the electrocardiogram generation unit 46.

Also, for example, the electrocardiographic information generation unit 45 or the electrocardiogram generation unit 46 may include a low pass filter, an amplifier, and an analog/digital converter. For example, unnecessary noise components are removed from a signal of the potential difference by the low pass filter, and the signal is amplified by the amplifier and then converted into a digital signal by the analog/digital converter.

The memory 47 includes a solid state drive (SSD), a random access memory (RAM), a read only memory (ROM), and the like. The memory 47 stores programs necessary for executing a variety of control processing. Further, the memory 47 stores the detected electrocardiographic signal, the generated electrocardiographic information and electrocardiogram information, and the like. Furthermore, for example, the memory 47 stores these pieces of information in a chronological order.

The control unit 48 includes one or a plurality of processors. The control unit 48 is formed of one or more processing circuits. The control unit 48 is, for example, a central processing unit (CPU). The control unit 48 causes the entire operation and predetermined operation (function) of the electrocardiographic measurement apparatus 1 to be executed based on the programs stored in the memory 47. The control unit 48 executes the predetermined operation, analysis, processing, or the like according to the read program. The control unit 48 controls operation of the operation unit 42, the display unit 43, the electrocardiographic information generation unit 45, and the electrocardiogram generation unit 46, transmits/receives a signal, and supplies power.

With the electrocardiographic measurement apparatus 1 configured as just described, the cap electrodes 32 are firstly mounted on the two of the plurality of base electrodes 31, suitably selected for electrocardiographic measurement, and a pair of cap electrodes 32 at a suitable distance is made. Therefore, a pair of electrodes 33 for detecting an electrocardiographic signal is made. Also, for example, the cap electrode 32 is mounted on any one of the remaining base electrodes 31, thereby forming the ground electrode 33A.

Then, the device body 12 is arranged on the upper arm 100, and the belt body 21 is fixed to the upper arm by the fixing means 23. Thus, the electrocardiographic measurement apparatus 1 is fixed to the upper arm of the subject. Then, the operation unit 42 is operated, and thus the control unit 48 controls each configuration and detects an electrocardiographic signal via the base electrodes 31 on which the cap electrodes 32 are disposed. Then, the electrocardiographic information generation unit 45 generates electrocardiographic information from the electrocardiographic signal, and the electrocardiogram generation unit 46 generates electrocardiogram information from the electrocardiographic information. The control unit 48 allows the memory 47 to store the electrocardiographic information and the electrocardiogram information and allows the display unit 43 to display information such as a date and time and an electrocardiogram. Also, the control unit 48 may control the communication unit to transmit a variety of information such as the date and time, the electrocardiographic information, and the electrocardiogram information to an external terminal.

Next, a method for deriving a pair of electrodes 33 at a suitable distance for each subject will be described. Note that the electrocardiographic measurement apparatus 1 may be used as a device for deriving the pair of electrodes 33 at a suitable distance for each subject. Also, another device that includes the same arrangement of electrodes as the arrangement of the plurality of base electrodes 31 of the electrocardiographic measurement apparatus 1 and that can perform electrocardiographic measurement may be used. Note that in the following description, the nine base electrodes 31 will be described as a first base electrode 311 to a ninth base electrode 319 in the arrangement order for explanatory convenience.

In the description of the method for deriving the pair of electrodes 33 of the present embodiment, an example in which the cap electrodes 32 are mounted on all of the base electrodes 311 to 319 of the electrocardiographic measurement apparatus 1 will be described. Further, the base electrodes 311 to 319 on which the cap electrodes 32 are mounted will be respectively described as the first electrode 311 to the ninth electrode 319.

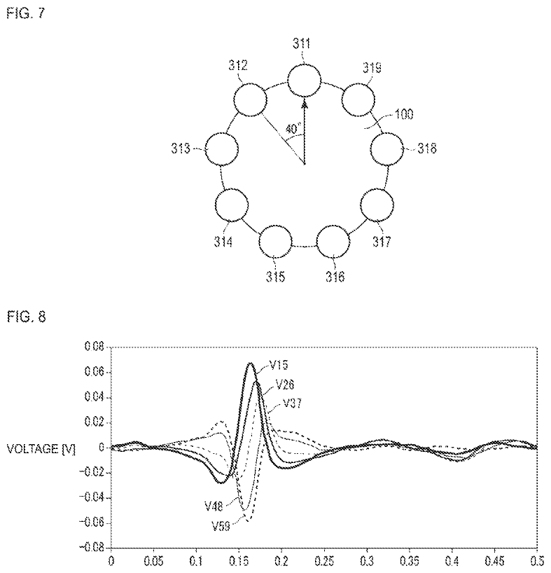

Furthermore, when the electrocardiographic measurement apparatus 1 is attached to the upper arm, the upper arm is oriented with the palm side of a hand facing upward. In addition, as illustrated in FIG. 7, assuming that the cross-sectional shape of the upper arm 100 is a circle, the first electrode 311 to the ninth electrode 319 will be described as in a state where the first electrode 311 to the ninth electrode 319 are located at a 40° interval in a counterclockwise direction.

First, a first example of the method for deriving the pair of electrodes 33 will be described with the use of FIGS. 7 and 8. FIG. 7 is an explanatory diagram illustrating, assuming that the cross-sectional shape of the upper arm 100 is a circle, the arrangement of the first electrode 311 to the ninth electrode 319. FIG. 8 is a graph illustrating an example of time series variations of electrocardiographic waveforms detected by respective pairs of electrodes 33.

The first example of the method for deriving the pair of electrodes 33 is a method for defining pairs of electrodes substantially at the same interval among the first electrode 311 to the ninth electrode 319, determining a time series variation of an electrocardiographic waveform obtained from a potential difference detected by each of the pairs of electrodes, and determining the intensity of the electrocardiographic waveform in each pair of electrodes.

As a specific example, for example, as illustrated in FIG. 7, the respective pairs of the first electrode 311 and the fifth electrode 315, the second electrode 312 and the sixth electrode 316, the third electrode 313 and the seventh electrode 317, the fourth electrode 314 and the eighth electrode 318, and the fifth electrode 315 and the ninth electrode 319 are defined from the first electrode 311 to the ninth electrode 319 of the electrocardiographic measurement apparatus 1 attached to the upper arm 100. Then, the electrocardiographic measurement apparatus 1 is operated to detect electrocardiographic signals corresponding to electric potentials of the respective pairs of electrodes, and electrocardiographic information is generated by the electrocardiographic information generation unit 45.

Thereafter, when electrocardiographic waveforms are generated by the electrocardiogram generation unit 46 from the electrocardiographic information, the electrocardiographic waveforms by the respective pairs of electrodes 33 are generated as illustrated in FIG. 8. FIG. 8 illustrates an example of time series variations of an electrocardiographic waveform V15 between the first electrode 311 and the fifth electrode 315, an electrocardiographic waveform V26 between the second electrode 312 and the sixth electrode 316, an electrocardiographic waveform V37 between the third electrode 313 and the seventh electrode 317, an electrocardiographic waveform V48 between the fourth electrode 314 and the eighth electrode 318, and an electrocardiographic waveform V59 between the fifth electrode 315 and the ninth electrode 319.

As illustrated in FIG. 8, the electrocardiographic waveforms generated by the respective pairs of electrodes 33 have different intensities. As can be seen, for example, the maximum peak intensity of the electrocardiographic waveform V15 between the first electrode 311 and the fifth electrode 315 is strongest, the intensity decreases in the order from the electrocardiographic waveform V26 between the second electrode 312 and the sixth electrode 316 to the electrocardiographic waveform V37 between the third electrode 313 and the seventh electrode 317, and the maximum peak intensities of the electrocardiographic waveform V48 between the fourth electrode 314 and the eighth electrode 318 and the electrocardiographic waveform V59 between the fifth electrode 315 and the ninth electrode 319 are at negative values.

In this case, the electrocardiographic waveform V15 between the first electrode 311 and the fifth electrode 315 is selected for electrocardiogram measurement, and the other pairs of electrodes 33 are not required. Therefore, in a case where the subject detects electrocardiogram information, the cap electrodes 32 are preferably mounted on the first base electrode 311 and the fifth base electrode 315 to make a pair of electrodes 33 for detecting an electrocardiographic signal, and then the cap electrode 32 for the ground electrode 33A is preferably mounted on any one of the other base electrodes 312, 313, 314, 316, 317, 318, 319. As just described, according to the first example of the method for deriving the pair of electrodes 33, the position of the electrode 33 at which the electrocardiographic signal detection intensity is high can be determined according to the subject.

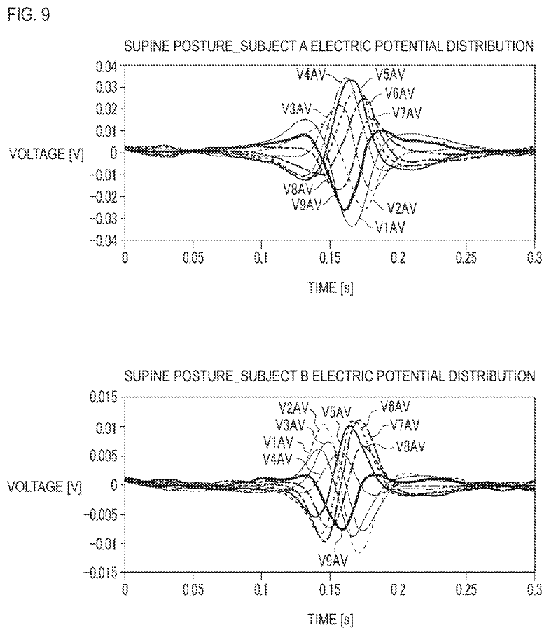

Next, a second example of the method for deriving the pair of electrodes will be described with the use of FIG. 9. FIG. 9 is a graph illustrating an example of time series variations of electrocardiographic waveforms detected by respective pairs of each of the electrodes 311 to 319 and a reference electrode in different subjects.

The second example is a method in which an average value of electric potential values between the first electrode 311 to the ninth electrode 319 and the reference electrode such as the ground electrode is determined as a reference electric potential, and time series variations of electrocardiographic waveforms obtained from potential differences each between the reference electric potential and an electric potential between the reference electrode and each of the first electrode 311 to the ninth electrode 319, then, the electrodes, the voltage peaks of which are at the plus side and at the negative side are defined as a pair of electrodes.

As a specific example, for example, the electrocardiographic measurement apparatus 1 attached to the upper arm 100 illustrated in FIG. 7 is operated to detect electrocardiographic signals corresponding to respective electric potentials from the first electrode 311 to the ninth electrode 319 and the reference electrode, and electrocardiographic information is generated by the electrocardiographic information generation unit 45. Additionally, the electrocardiographic information generation unit 45 or the control unit 48 obtains an average value (AV) of the electrocardiographic signals corresponding to the respective electric potentials. Next, electrocardiographic waveforms are generated by the electrocardiogram generation unit 46 from the respective electric potentials of the ground electrode 33A and the first electrode 311 to the ninth electrode 319 and the average value (reference electric potential). Thus, as illustrated in FIG. 9, the electrocardiographic waveforms by the respective pairs of each of the electrodes 311 to 319 and the average value of the electrocardiographic signals are generated. In FIG. 9, an example of time series variations of an electrocardiographic waveform V1AV of the first electrode 311 and the average value, an electrocardiographic waveform V2AV of the second electrode 312 and the average value, an electrocardiographic waveform V3AV of the third electrode 313 and the average value, an electrocardiographic waveform V4AV of the fourth electrode 314 and the average value, an electrocardiographic waveform V5AV of the fifth electrode 315 and the average value, an electrocardiographic waveform V6AV of the sixth electrode 316 and the average value, an electrocardiographic waveform V7AV of the seventh electrode 317 and the average value, an electrocardiographic waveform V8AV of the eighth electrode 318 and the average value, and an electrocardiographic waveform V9AV of the ninth electrode 319 and the average value. Further, the electrocardiographic waveforms V1AV to V9AV of each of different subjects (A and B) are illustrated in FIG. 9. Furthermore, the posture of the subjects at the time of generating the electrocardiographic waveforms is the supine posture.

As illustrated in FIG. 9, the electrocardiographic waveforms V1AV to V9AV differ in intensity at the positive side and the negative side of the voltage. For example, in the case of the subject A, the peak intensity of the electrocardiographic waveform V4AV of the fourth electrode 314 and the average value is largest at the positive side of the voltage, and the peak intensity of the electrocardiographic waveform V1AV of the first electrode 311 and the average value is largest at the negative side of the voltage. Accordingly, by making a pair of the first electrode 311 and the fourth electrode 314, it can be estimated that the maximum peak intensity of the electrocardiographic waveform between the first electrode 311 and the fourth electrode 314 is highest.

Consequently, in the case of the subject A, the first electrode 311 and the fourth electrode 314 are selected for electrocardiogram measurement, and the other electrodes 33 are not required. As a result, in a case where the subject A subsequently detects electrocardiogram information, the cap electrodes 32 are mounted on the first base electrode 311 and the fourth base electrode 314, thereby forming the electrodes 33 for detecting electrocardiographic signals. In addition, the cap electrode 32 for the ground electrode 33A is mounted on any one of the other base electrodes 312, 313, 315, 316, 317, 318, 319.

As can be seen from FIG. 9, even when the electrode 33 is disposed at the same position on the upper arm 100 in the case of the subject A and the case of the subject B, the peak intensity and the peak position of the electrocardiographic waveform differ between the subjects. For example, in the case of the subject B, the peak intensity of the electrocardiographic waveform V6AV of the sixth electrode 316 and the average value is largest at the positive side of the voltage, and the peak intensity of the electrocardiographic waveform V2AV of the second electrode 312 and the average value is largest at the negative side of the voltage. Accordingly, by making a pair of the second electrode 312 and the sixth electrode 316, it can be estimated that the maximum peak intensity of the electrocardiographic waveform between the second electrode 312 and the sixth electrode 316 is highest.

Consequently, in the case of the subject B, the second electrode 312 and the sixth electrode 316 are selected for electrocardiogram measurement, and the other electrodes 33 are not required. As a result, in a case where the subject B subsequently detects electrocardiogram information, the cap electrodes 32 for detecting electrocardiographic signals are preferably mounted on the second base electrode 312 and the sixth base electrode 316, and the cap electrode 32 for the ground electrode 33A is preferably mounted on any one of the other base electrodes 31. As described above, according to the second example, a pair of suitable electrodes 33 can be determined according to the subject.

Additionally, examples of the electrocardiographic waveforms of the different subjects A and B as subjects in the second example illustrated in FIG. 9 are illustrated; however, the electric potential distributions of the electrocardiograms obtained in the respective subjects are different therebetween. Consequently, by deriving a pair of electrode positions suitable for the subject and selecting the base electrode 31 for mounting thereon the cap electrode 32, two positions of the electrodes 33, the maximum peak intensity of which is high can be obtained with a small number of cap electrodes 32.

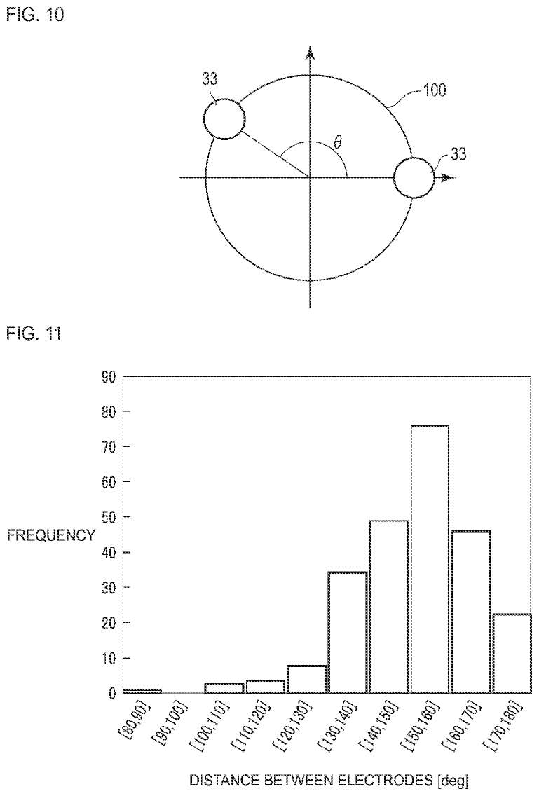

Next, the intensity of potential difference in a case where the distance between the two electrodes 33 on the upper arm 100 is changed will be described with the use of FIGS. 10 and 11. FIG. 10 is an explanatory diagram illustrating an example of the distance between the two electrodes 33 on the upper arm 100. FIG. 11 illustrates a histogram of the distance between the electrodes 33 where the potential difference is largest. As illustrated in FIG. 10, assuming that the cross-section of the upper arm 100 has an approximately circle shape, the electrode is placed at one point from the center on the surface of the upper arm 100, and another electrode is disposed at an intersection between a straight line having a predetermined angle θ with respect to a straight line connecting from the center to the one point of the upper arm surface, and the upper arm surface. In addition, when the angle θ is set to be large, the distance between the electrodes is increased.

For example, as illustrated in the example illustrated in FIG. 11, when the distance between the electrodes, i.e., the angle that maximizes the potential difference is determined from the histogram of the electrode distance where the maximum potential difference is attained, the average angle is 156.4 degrees, and the standard deviation is 15.2 degrees. In other words, it is preferable that the angle be a 140 to 170 degree interval. Therefore, for example, in a case where one pair of electrodes is defined in the first example, the angle is preferably defined within the range of a 140 to 170 degree interval. Also, in the second example, a pair of electrodes having the angle at a 140 to 170 degree interval is preferably selected.

According to the belt 11 and the electrocardiographic measurement apparatus 1 that are configured as just described, the plurality of base electrodes 31 more than the number of electrodes that is required to generate electrocardiogram information are provided, and the cap electrodes 32 for detecting electrocardiographic signals are selectively mounted on the base electrodes 31. Consequently, the cap electrodes 32 are mounted on the base electrodes 31 that can make a pair of electrodes 33, the maximum peak intensity of which is high, and thus the electrocardiographic measurement apparatus 1 can suitably detect an electrocardiographic signal with a small number of electrodes.

As a result, even in a case where electrocardiogram information of subjects, the upper arms 100 of which have different arm circumference lengths is generated, a pair of suitable electrodes 33 is obtained in advance for each subject, and thus the electrocardiogram information can be easily generated by the pair of suitable electrodes 33. Further, by determining in advance the pair of suitable electrodes 33 for each subject, the cap electrodes 32 can be disposed at the positions at which the electrocardiographic signal detection intensity is high. Therefore, the electrocardiographic measurement apparatus 1 can improve the electrocardiographic signal detection intensity.

Furthermore, the cap electrodes 32 only need to be disposed on at least any two of the plurality of base electrodes 31 and on three of the plurality of base electrodes 31 when the ground electrode 33A is configured. Accordingly, the electrocardiographic measurement apparatus 1 can generate electrocardiographic information and electrocardiogram information by a pair of cap electrodes 32, and thus can reduce the processing required to generate the electrocardiographic information and electrocardiogram information.

In other words, many electrodes are used as in a conventional electrocardiographic measurement apparatus in order to enable electrocardiogram information to be generated even in any of a plurality of subjects. In such a case, it is necessary that the electrocardiographic information and the electrocardiogram information are generate by respective pairs of electrodes. Therefore, in the conventional electrocardiographic measurement apparatus, the processing for generating the electrocardiogram information requires time and consumed power, and a circuit for generating the electrocardiogram information is complicated.

However, according to the electrocardiographic measurement apparatus 1 according to one embodiment of the present invention, the electrocardiographic information and electrocardiogram information may be generated by only one pair of electrodes 33. As a result, the electrocardiographic measurement apparatus 1 can suppress time and consumed power for processing and can prevent the circuit for generating electrocardiographic waveforms from being complicated.

Additionally, the base electrode 31 and the cap electrode 32 that form the electrode 33 are configured detachably and attachably, and thus the position of the cap electrode 32 can be easily changed. Moreover, the base electrode 31 and the cap electrode 32 are formed in a snap button shape such that one and the other are provided with the spring 31b and the stud 32b, respectively. Therefore, the cap electrode 32 can be more easily attached and detached.

Further, since the base electrodes 31 are fixed to the belt body 21, only the cap electrodes 32 can be selectively mounted on the base electrodes 31. Therefore, in order to change the electrode 33 to be brought in contact with the upper arm 100, the cap electrode 32 only need to be shifted in the belt 11, and the position of the electrode 33 can be easily changed. Furthermore, the stud 32b of one of the base electrode 31 and the cap electrode 32 is inserted into the recess 31a of the other of the base electrode 31 and the cap electrode 32, and the stud 32b is held by the spring 31b. Thus, the base electrode 31 and the cap electrode 32 can be easily fixed.

As described above, according to the electrocardiographic measurement apparatus 1 according to the first embodiment, an electrocardiographic signal can be suitably detected with a small number of electrodes.

### Other Embodiments

A plurality of embodiments will be described below as an electrocardiographic measurement apparatus according to other embodiments of the present invention. Note that each of other embodiments is different from the electrocardiographic measurement apparatus 1 according to the first embodiment described above in the configuration of the electrode array 22 of the belt 11, and thus only the configuration of the belt is illustrated and the description of the configuration of the device body 12 is omitted. Additionally, of the configurations of the belt and the electrocardiographic measurement apparatus according to other embodiments, the same configurations as those in the electrocardiographic measurement apparatus 1 according to the first embodiment described above are denoted by the same reference numerals, and descriptions thereof are omitted.

### Second Embodiment

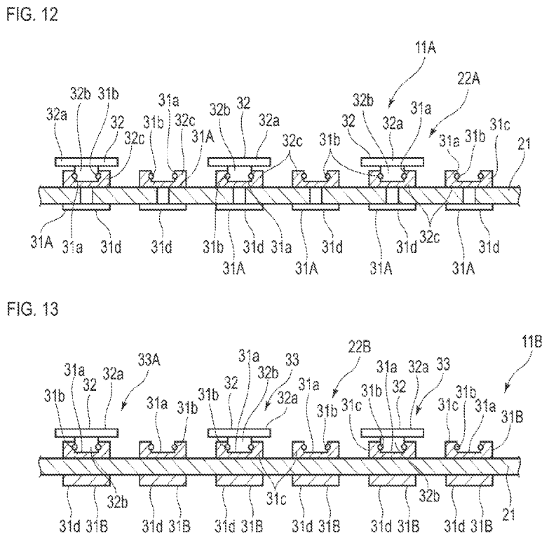

A belt 11A according to a second embodiment will be described with the use of FIG. 12. Note that FIG. 12 is a cross-sectional view schematically illustrating the configuration of the belt 11A.

As illustrated in FIG. 12, the belt 11A includes the belt body 21 and an electrode array 22A.

The electrode array 22A is electrically connected via a signal line or the like to the device body 12. The electrode array 22A includes a plurality of base electrodes 31A and the plurality of cap electrodes 32.

The plurality of base electrodes 31A are disposed side by side at equal intervals in the longitudinal direction of the belt body 21. Each of the plurality of base electrodes 31A is electrically connected to the device body 12. The base electrode 31A is formed to enable the cap electrode 32 to be attached and detached.

The number of base electrodes 31A can be appropriately set to the number that can generate an electrocardiographic waveform even when the upper arms of subjects on which the measurement of electrocardiographic waveforms is expected have different arm circumference lengths. In other words, in order to accommodate the upper arms of a variety of subjects, the number of base electrodes 31A is set to the number more than the number of electrodes required to generate an electrocardiographic waveform. For example, nine base electrodes 31A are disposed. Note that in FIG. 12, the base electrodes 31A are illustrated while partially omitted.

A specific example of such base electrode 31A and cap electrode 32 is illustrated below. In order to enable the cap electrode 32 to be detached and attached to the base electrode 31A, the base electrode 31A and the cap electrode 32 are insertably formed. For example, one of the base electrode 31A and the cap electrode 32 includes a spring, and the other is formed in a snap button shape that includes a stud.

As a specific example, the base electrode 31A includes the recess 31a and the spring 31b disposed in the recess 31a. Additionally, the base electrodes 31A are fixed to the belt body 21 by inserting or crimping. For example, the base electrode 31A includes a body 31c that includes the recess 31a and the spring 31b, and a post 31d with which the body 31c is fixed to the belt body 21. Additionally, the body 31c and the post 31d are disposed on both main surfaces of the belt body 21, and the post 31d is crimped to the body 31c; therefore, the base electrode 31A is fixed to the belt body 21.

The belt 11A configured as just described exerts the same effects as the belt 11 according to the first embodiment described above. In addition, the belt 11A is configured such that the body 31c and the post 31d are crimped with the belt body 21 interposed therebetween, and the base electrode 31A can be firmly fixed to the belt body 21.

### Third Embodiment

A belt 11B according to a third embodiment will be described with the use of FIG. 13. Note that FIG. 13 is a cross-sectional view schematically illustrating the configuration of the belt 11B.

As illustrated in FIG. 13, the belt 11B includes the belt body 21 and an electrode array 22B.

The electrode array 22B is electrically connected via a signal line or the like to the device body 12. The electrode array 22B includes a plurality of base electrodes 31B and the plurality of cap electrodes 32.

The plurality of base electrodes 31B are disposed side by side at equal intervals in the longitudinal direction of the belt body 21. Each of the plurality of base electrodes 31B is electrically connected to the device body 12. The base electrode 31B is formed to enable the cap electrode 32 to be attached and detached.

The number of base electrodes 31B can be appropriately set to the number that can generate an electrocardiographic waveform even when the upper arms of subjects on which the measurement of electrocardiographic waveforms is expected have different arm circumference lengths. In other words, in order to accommodate the upper arms of a variety of subjects, the number of base electrodes 31B is set to the number more than the number of electrodes required to generate an electrocardiographic waveform. For example, nine base electrodes 31B are disposed.

Note that in FIG. 13, the base electrodes 31B are illustrated while partially omitted.

A specific example of such base electrode 31B and cap electrode 32 is illustrated below. In order to enable the cap electrode 32 to be detached and attached to the base electrode 31B, the base electrode 31B and the cap electrode 32 are insertably formed. For example, one of the base electrode 31B and the cap electrode 32 includes a spring, and the other is formed in a snap button shape that includes a stud.

As a specific example, the base electrode 31B includes the recess 31a and the spring 31b disposed in the recess 31a. Also, the base electrode 31B is fixed to the belt body 21 by magnetic force. For example, the base electrode 31B includes the body 31c that includes the recess 31a and the spring 31b, and the post 31d with which the body 31c is fixed to the belt body 21. Additionally, the body 31c and the post 31d are formed of magnets or include magnets. The body 31c and the post 31d include different magnetic poles in a region facing each other, and are stuck to each other. The body 31c and the post 31d are disposed on the both main surfaces of the belt body 21, and the base electrode 31B is fixed to the belt body 21 by the magnetic force of the body 31c and the post 31d. Note that one of the body 31c and the post 31d may be formed of a magnet or include a magnet, and the other may be formed of a magnetic metal material.

The belt 11B configured as just described exerts the same effects as the belt 11 according to the first embodiment described above. In addition, the base electrode 31B can be fixed to the belt body 21 by the magnetic force.

### Fourth Embodiment

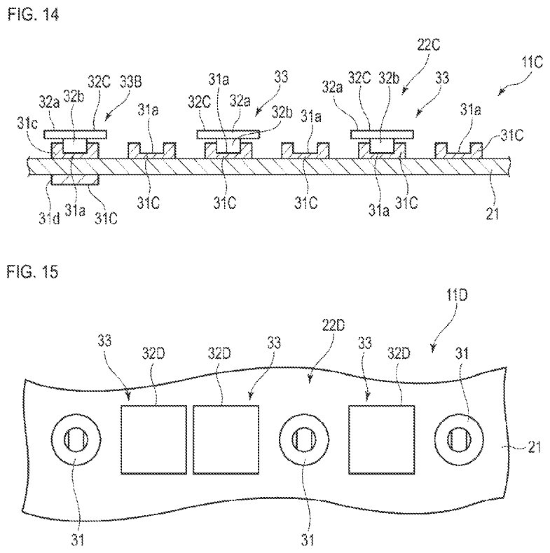

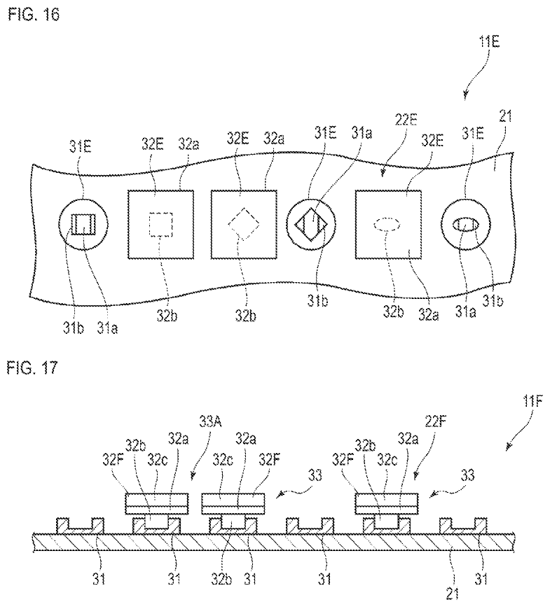

A belt 11C according to a fourth embodiment will be described with the use of FIG. 14. Note that FIG. 14 is a cross-sectional view schematically illustrating the configuration of the belt 11C.

As illustrated in FIG. 14, the belt 11C includes the belt body 21 and an electrode array 22C.

The electrode array 22C is electrically connected via a signal line or the like to the device body 12. The electrode array 22C includes a plurality of base electrodes 31C and a plurality of cap electrodes 32C.

The plurality of base electrodes 31C are disposed side by side at equal intervals in the longitudinal direction of the belt body 21. Each of the plurality of base electrodes 31C is electrically connected to the device body 12. The base electrode 31C is formed to enable the cap electrode 32C to be attached and detached.

The number of base electrodes 31C can be appropriately set to the number that can generate an electrocardiographic waveform even when the upper arms of subjects on which the measurement of electrocardiographic waveforms is expected have different arm circumference lengths. In other words, in order to accommodate the upper arms of a variety of subjects, the number of base electrodes 31C is set to the number more than the number of electrodes required to generate an electrocardiographic waveform. For example, nine base electrodes 31C are disposed.

Note that in FIG. 14, the base electrodes 31C are illustrated while partially omitted.

A specific example of such base electrode 31C and cap electrode 32C is illustrated below. In order to enable the cap electrode 32C to be detached and attached to the base electrode 31C, the base electrode 31C and the cap electrode 32C are formed so as to be fixed by magnetic force. For example, one of the base electrode 31C and the cap electrode 32C includes a recess, and the other includes a stud.

Additionally, the base electrode 31C and the cap electrode 32C include different magnetic poles in a region facing each other, and the stud is inserted into the recess. Thus, the base electrode 31C and the cap electrode 32C are stuck to each other to be fixed.

As a specific example, the base electrode 31C includes the recess 31a. Further, the base electrode 31C is fixed, for example, by affixing, sewing, inserting, crimping, or magnetic force. For example, the base electrodes 31C are fixed to the belt body 21 by affixing or sewing as illustrated from the second from the left to the first from the right in FIG. 14. Note that, as illustrated at the first from the left in FIG. 14, the base electrode 31C includes the body 31c and the post 31d. The body 31c and the post 31d may be formed of magnets or include magnets, and include different magnetic poles in a region facing each other, and may be stuck to each other. In other words, the electrode 33 may be configured to fix the body 31c and the post 31d by the magnetic force and fix the body 31c and the cap electrode 32C by the magnetic force.

The belt 11C configured as just described exerts the same effects as the belt 11 according to the first embodiment described above. In addition, since the base electrode 31C and the cap electrode 32C are configured to stick to each other by the magnetic force, the cap electrode 32C may be mounted on the base electrode 31C only by bringing the cap electrode 32C close to the base electrode 31C. Thus, mountability of the cap electrode 32C can be improved.

### Other Embodiments

Note that the present invention is not limited to the embodiments described above. For example, in the example described above, an example is described in which the cap electrode 32 is configured such that the surface, which is brought into contact with the living body, of the electrode portion 32a is formed, for example, in a circular planer shape, but is not limited thereto. For example, as in a cap electrode 32D used in an electrode array 22D of a belt 11D according to a fifth embodiment illustrated in FIG. 15, the surface may be formed in planer shape of a polygonal shape, specifically, a rectangular shape, and more specifically, a square shape or a box shape. The surface is formed in a polygonal shape and more preferably in a rectangular shape, and thus the area of a surface, which is brought into contact with the living body, of the electrode portion 32a of the cap electrode 32D can be increased compared to a circular shape. In particular, the electrode 33 (cap electrode 32D) is used to acquire an electrocardiographic signal, and in order to ensure the required signal/noise ratio, the size that is brought into contact with the living body is desirably large in the electrode 33. In addition, the suitable surface shape of the electrode portion 32a of the cap electrode 32D that can increase the size of the electrode 33 is typically a square shape or box shape. Therefore, by forming the surface shape of the electrode portion 32a of the cap electrode 32D in a rectangular shape, the size that is brought into contact with the living body can be increased in the electrode 33, and the required signal/noise ratio is easily ensured.

In addition, for example, the surface shape of the electrode portion 32a of the cap electrode 32D is formed in a rectangular shape as in the belt 11D described in the fifth embodiment illustrated in FIG. 15. Accordingly, in a case where the cap electrodes 32D are disposed adjacent to each other, the cap electrode 32D rotates around the axis of the stud 32b and thus the adjacent cap electrodes 32D may interfere with each other. Therefore, for example, as illustrated in a base electrode 31E and a cap electrode 32E of an electrode array 22E of a belt 11E described in a sixth embodiment, the recess 31a disposed in one of the base electrode 31E and the cap electrode 32E, and the stud 32b disposed on the other of the base electrode 31E and the cap electrode 32E may be configured to include an open cross-sectional shape or a cross-sectional shape other than a circular shape.

In other words, the shape of the recess 31a and the stud 32b in a planar view may be a shape other than a circular shape, such as a rectangular shape including a square shape, a box shape, or a trapezoidal shape or such as an elliptical shape, a star shape, or a crisscross shape. When the cap electrode 32E is mounted on the base electrode 31E in a predetermined position, the recess 31a and the stud 32b interfere around the axis, and thus the belt 11E configured as just described can prevent the base electrode 31E from rotating around the axis of the stud 32b. As a result, the adjacent cap electrodes 32E can be prevented from being brought into contact with each other. Additionally, the position of the stud 32b when inserting the stud 32b into the recess 31a is limited, and thus the position of the cap electrode 32E is also limited. Therefore, only by simply inserting the stud 32b into the recess 31a, the cap electrode 32E can be mounted on the base electrode 31E in a suitable position.

Further, in the example described above, an example in which the cap electrode 32 is a dry electrode is described, but is not limited thereto. For example, the cap electrode may be a wet electrode. In the case of the wet electrode, a conductive wet member 32c may be disposed on the surface on the living body side of the electrode portion 32a as in a cap electrode 32F of an electrode array 22F of a belt 11F according to a seventh embodiment illustrated in FIG. 17. For example, the wet member 32c can be set as appropriate as long as the wet member 32c is made of a material that can reduce surface resistance between the surface of the electrode portion 32a of the electrode 33, 33A (cap electrode 32F) such as conductive gel sheet and the living body surface and that can increase a signal/noise ratio of an electrocardiographic signal.

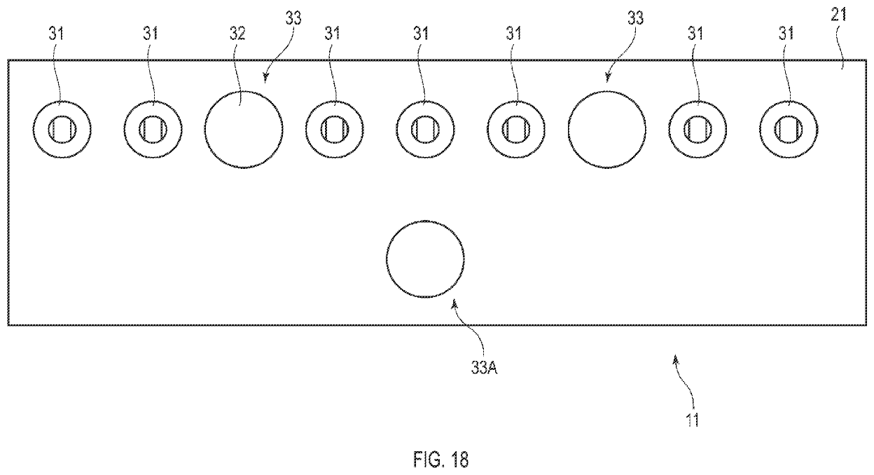

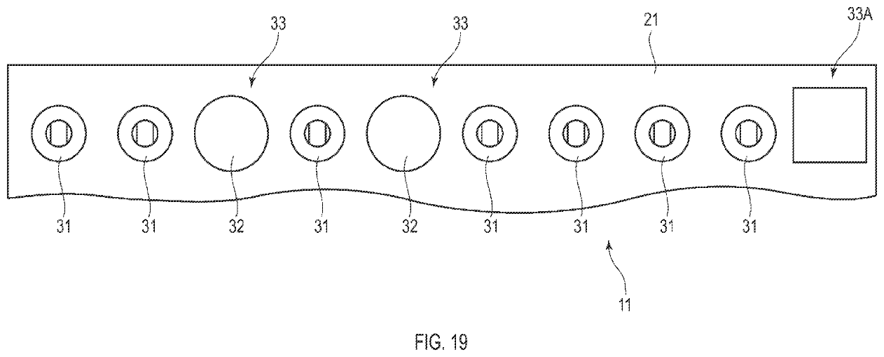

In addition, in the example described above, an example is described in which three cap electrodes 32 are mounted on the selected base electrodes 31, and electrocardiographic signals are detected by two cap electrodes 32 and one cap electrode 32 is applied as the ground electrode 33A, but is not limited thereto. For example, as illustrated in FIGS. 18 and 19, instead of mounting the cap electrode 32 on the base electrode 31, the ground electrode 33A may be disposed on the belt 11 in advance. In this case, as illustrated in FIG. 18, the ground electrode 33A may be disposed at a position shifted from the base electrode 31 in a direction orthogonal to the arrangement direction of the base electrodes 31. Also, as illustrated in FIG. 19, the ground electrode 33A may be disposed side by side with the base electrode 31 disposed at one end in the arrangement direction of the plurality of arranged base electrodes 31. In addition, the shape of the electrode portion of such a ground electrode 33A may be a circular shape, may be a rectangular shape, or may be a polygonal shape other than a rectangular shape. Moreover, the belt 11 may be configured without a ground electrode.

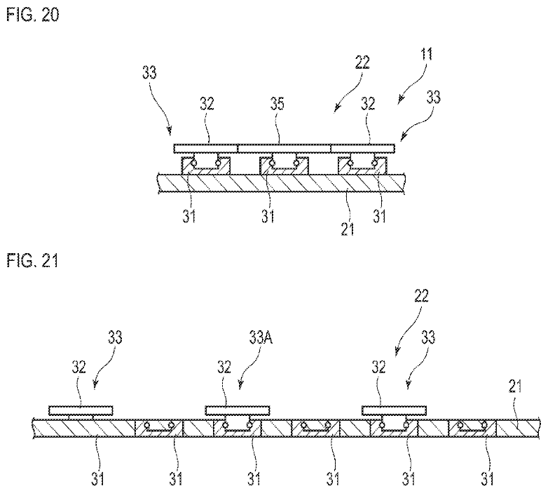

Further, in the example described above, the configuration where the base electrode 31 on which the cap electrode 32 is not mounted is exposed to the outside is described, but is not limited thereto. For example, as illustrated in FIG. 20, a cap 35 formed of a non-conductive material may be disposed on the base electrode 31 on which the cap electrode 32 is not mounted. Furthermore, in the example described above, the configuration where the base electrodes 31 are disposed on the main surface on the living body side of the belt body 21 is described, but is not limited thereto. As illustrated in FIG. 21, the base electrodes 31 may be embedded in the belt body 21.

Additionally, in the example described above, the configuration where the base electrode 31 includes the recess 31a and the cap electrode 32 includes the stud 32b is described, but is not limited thereto. The base electrode 31 may include the stud 32b and the cap electrode 32 may include the recess 31a.

Moreover, in the example described above, a variety of configuration examples in which the base electrode 31 and the cap electrode 32 include the recess 31a and the stud 32b are described, but is not limited thereto. For example, the recess 31a and the stud 32b include an internal thread and an external thread to be screwed with each other, and thus the recess 31a and the stud 32b may be fixed. In addition, in the example in which the base electrode 31 and the cap electrode 32 are fixed by the magnetic force, the base electrode 31 and the cap electrode 32 may be configured without the recess 31a, the spring 31b, and the stud 32b.

Further, in the aforementioned example, the electrocardiographic measurement apparatus 1 is described with the use of an example in which the belt 11 is attached to the upper arm but may be configured to be attached to the chest or to other portions of the living body.

Furthermore, in the aforementioned examples, the configuration where the belt 11 is used in the electrocardiographic measurement apparatus 1 is described, but is not limited thereto. For example, the belt 11 may be used in a biological information measurement device used in electrocardiographic measurement and blood pressure measurement. As a specific example, in addition to the configuration of the electrocardiographic measurement apparatus 1 described above, the biological information measurement device may include a processing circuit or the like that exerts a function of blood pressure measurement, which generates a blood pressure value from a pulse wave sensor and pulse wave information detected by the pulse wave sensor. Such a biological information measuring device calculates a pulse transit time (PTT) per heartbeat and achieve a function to estimate a blood pressure value. Note that such a biological information measurement device calculates the pulse transit time

(PTT) per heartbeat based on, for example, a time difference between an R-wave peak RP detected by an electrocardiographic signal and a pulse wave rise PS per heartbeat, which is one of the feature values of a pulse wave signal detected by the pulse wave sensor.

While the embodiments according to the present invention have been described in detail above, the above-described description merely exemplifies the present invention in all respects, and obviously, various improvements and modifications can be made without departing from the scope of the present invention. That is, specific configurations according to the respective embodiment may be employed as appropriate in the implementation of the present invention.

Additionally, in the present invention, various inventions can be formed by appropriately combining a plurality of components disclosed in the embodiments described above. For example, some components may be omitted from all the components described in the respective embodiments. Further, the components of the different embodiments may be combined appropriately.

### REFERENCE NUMERALS LIST

1 Electrocardiographic measurement apparatus

11 Belt

11A Belt

11B Belt

11C Belt

11D Belt

11E Belt

11F Belt

12 Device body

21 Belt body

22 Electrode array

22A Electrode array

22B Electrode array

22C Electrode array

22D Electrode array

22E Electrode array

22F Electrode array

23 Fixing means

31 Base electrode

31A Base electrode

31b Spring

31B Base electrode

31c Body

31C Base electrode

31d Post

31E Base electrode

32 Cap electrode

32a Electrode portion

32b Stud

32c Wet member

32C Cap electrode

32D Cap electrode

32E Cap electrode

32F Cap electrode

33 Electrode

33A Ground electrode

35 Cap

41 Case

42 Operation unit

42a Button

43 Display unit

44 Power supply unit

45 Electrocardiographic information generation unit

46 Electrocardiogram generation unit

47 Memory

48 Control unit

100 Upper arm

What is claimed is:

1. A belt, comprising:
a belt body to be wrapped around a living body;
three or more base electrodes disposed in a longitudinal direction of the belt body; and
two or more cap electrodes detachable and attachable to the base electrodes and smaller in number than the number of base electrodes.

2. The belt according to claim 1, wherein
the base electrodes are fixed to the belt body by affixing, sewing, inserting, crimping, or magnetic force, and
the cap electrodes are fixed to the base electrodes selectively and detachably and attachably by inserting, magnetic force, or screwing.

3. The belt according to claim 1, wherein the cap electrodes include surfaces that are brought into contact with the living body, the surfaces being formed in a circular shape or a polygonal shape.

4. The belt according to claim 1, wherein one of the base electrode and the cap electrode includes a recess, and another of the base electrode and the cap electrode includes an insertion portion to be inserted into the recess.

5. The belt according to claim 4, wherein the recess and the insertion portion are shaped to restrict rotation of the insertion portion around an axis of the insertion portion.

6. The belt according to claim 1, wherein the cap electrode is a dry electrode or a wet electrode.

7. An electrocardiographic measurement apparatus, comprising:
the belt according to claim 1; and
a device body configured to detect an electrocardiographic waveform by the cap electrodes mounted on the base electrodes.

8. The belt according to claim 2, wherein the cap electrodes include surfaces that are brought into contact with the living body, the surfaces being formed in a circular shape or a polygonal shape.

9. The belt according to claim 2, wherein one of the base electrode and the cap electrode includes a recess, and another of the base electrode and the cap electrode includes an insertion portion to be inserted into the recess.

10. The belt according to claim 3, wherein one of the base electrode and the cap electrode includes a recess, and another of the base electrode and the cap electrode includes an insertion portion to be inserted into the recess.

11. The belt according to claim 8, wherein one of the base electrode and the cap electrode includes a recess, and another of the base electrode and the cap electrode includes an insertion portion to be inserted into the recess.

12. The belt according to claim 9, wherein the recess and the insertion portion are shaped to restrict rotation of the insertion portion around an axis of the insertion portion.

13. The belt according to claim 10, wherein the recess and the insertion portion are shaped to restrict rotation of the insertion portion around an axis of the insertion portion.

14. The belt according to claim 11, wherein the recess and the insertion portion are shaped to restrict rotation of the insertion portion around an axis of the insertion portion.

15. The belt according to claim 2, wherein the cap electrode is a dry electrode or a wet electrode.

16. The belt according to claim 3, wherein the cap electrode is a dry electrode or a wet electrode.

17. The belt according to claim 4, wherein the cap electrode is a dry electrode or a wet electrode.

18. The belt according to claim 8, wherein the cap electrode is a dry electrode or a wet electrode.

19. The belt according to claim 9, wherein the cap electrode is a dry electrode or a wet electrode.

20. The belt according to claim 10, wherein the cap electrode is a dry electrode or a wet electrode.
